(19) United States
(12) Patent Application Publication
(10) Pub. No.: US 2003/0186403 A1
(43) Pub. Date: Oct. 2, 2003

(54) Preparation of immobilised acclimated micro-organisms, production method and use for preactivating interrupted permentation processes

(76) Inventors: Maria De Fatima Teixeira Da Silva, Gulpilhares (PT); Leonor Santos, Vila Real (PT); Laurent Dulau, Toulouse (FR)

Correspondence Address: YOUNG & THOMPSON, 745 SOUTH 23RD STREET 2ND FLOOR, ARLINGTON, VA, 22202

(21) Appl. No.: 10/343,759

(22) PCT Filed: Aug. 3, 2001
(86) PCT No.: PCT/FR01/02537

(30) Foreign Application Priority Data
Aug. 4, 2000 (FR) 00/10319

(51) Int. Cl. C12P 7/06; C12N 1/18

(52) U.S. Cl. 435/161; 435/254.21

(57) ABSTRACT

The invention concerns a preparation of micro-organisms immobilised in beads acclimated to alcohol and/or acidity, and partly dried, capable of developing a fermenting activity when they are introduced in alcohol or acid musts. The invention also concerns a method for obtaining such a preparation comprising an acclimating step and an immobilising step for the micro-organisms, said steps being carried out in any sequence, but necessarily preceding a partial dehydrating step. The invention provides the advantages on enabling the micro-organisms to be preserved in viable and active form for several months, and to be used directly for reactivating interrupted fermenting processes, in particular alcoholic fermentation processes, for producing wine and other fermented beverages.

The present invention relates to a preparation of micro-organisms, immobilised in semi-humid beads, capable of ensuring the re-starting of fermentation in fermented drinks.

It is known that immobilised micro-organisms may be used in the production of fermented drinks such as wine, beer, champagne and sparkling drinks with a variable degree of alcohol.

Various methods of immobilising cells are known and, in particular, the following may be mentioned:

The patent application EP 0350374 A1, which describes the preparation of micro-organisms immobilised in substantially dehydrated gels, the use of which is intended for preparing fermented drinks.

The patent application EP 0173915 B1, which describes the preparation of a bio-catalyst with cells immobilised in a gel for the fermentation and/or the formation of bubbles in sparkling wine or wine obtained in accordance with the champagne method.

The patent FR 2570959, which describes the method of obtaining beads containing micro-organisms used for the fermentation of the drinks mentioned above.

In all of these techniques, the micro-organisms are immobilised in polymer matrices and introduced into the must from the start of the fermentation process, whilst the conditions of the medium are favourable to their metabolic activity. However, it often happens that fermentation is retarded, or even stopped, before the sugar and alcohol and/or acid contents have reached the desired levels. It is then necessary to reactivate fermentation by a new introduction of micro-organisms.

The problem posed at present is to provide a tool which permits interrupted fermentation to be reactivated without tedious acclimatisation steps for the micro-organisms. In fact, after the unintentional arrest of a fermentation in progress, the addition of micro-organisms, for example yeast in the case of alcoholic fermentation processes, to reactivate fermentation requires a step called the acclimatisation of said micro-organisms to the conditions of the fermentation medium (in particular because of the concentration of alcohol and/or of acid already produced); in the absence of such a step, the non-acclimatised micro-organisms are subjected to stress which generally causes them to lose their viability, and hence the attempt to reactivate fermentation fails. As a consequence, techniques used at present have to choose between two methods.

A first method uses micro-organisms in the dry form, which are rehydrated, then progressively acclimatised, before being introduced into the must. Acclimatisation consists of subjecting the micro-organisms to incubation which can last between a few hours and several days, in media which have an increasing concentration of alcohol and/or acidity, and this occurs just before they are used.

A second method uses micro-organisms which have been previously acclimatised away from the place of use. They are transported in humid form and introduced in that form into the must. In this last case, they must be used within a brief period of a few days, since their preservation period is short.

However it has not been possible until now to combine the advantages associated with the two techniques: dried cells and acclimatised cells. In fact, the drying of micro-organisms, which generally permits them to be preserved well and transported easily, is not applicable to acclimatised micro-organisms, more especially yeasts, since it causes great and rapid mortality in this case.

The present invention relates to a preparation of micro-organisms which has, at one and the same time, the interesting characteristics of the micro-organisms in the dried form and of the acclimatised micro-organisms. In fact, it has been found, surprisingly, that micro-organisms immobilised and acclimatised in partially dried polymer beads permitted, at one and the same time, said micro-organisms to be very viable and to retain their ability to reactivate interrupted fermentation, and this even after a prolonged storage of several months.

Thus, the present invention relates to preparations of micro-organisms, immobilised in polymer beads and acclimatised to alcohol and/or to acidity, which are capable of ensuring that interrupted fermentation processes are reactivated. The present invention also relates to a method of obtaining such preparations of micro-organisms.

The preparation of micro-organisms according to the invention may be achieved with any genus of living micro-organism capable of being immobilised and acclimatised to alcohol and/or to acidity. Such micro-organisms are described in literature and are well-known to the person skilled in the art. According to a preferred mode of implementation of the present invention, the immobilised micro-organisms are yeasts, preferably of the Saccharomyces genus, such as Saccharomyces cerevisiae.

The beads in which the micro-organisms are immobilised are made up of reticulated polymers which form a matrix, said polymers being compatible with good viability of the micro-organisms and with their use in food applications. Such polymers may be polysaccharides, preferably a calcium alginate gel. The alginate is a linear polymer extracted from algae and made up of α-D-manuronic and α-L-guluronic acid. Matrices of polyacrylamide, pectate or carraghenane may also be used, for example.

Particles of spherical form or of any other form, obtained by splitting the polymer matrix enclosing the micro-organisms, are called “beads”. According to one of the characteristics of the present invention, the average size of these particles is between 0.1 and 5 mm, preferably between 1 and 3 mm. According to another characteristic of the invention, the proportion of micro-organisms relative to the polymer is between 1 and 50%, preferably between 5 and 20% dry weight.

According to another characteristic of the invention, the humidity of the polymer beads is monitored, so that they have a dry appearance but contain a sufficient proportion of water to maintain the viability of the micro-organisms. The humidity is determined by measuring the activity of the water Aw in the beads, said activity being between 0.1 and 0.5, and preferably between 0.3 and 0.4.

According to one characteristic of the invention, the immobilised living micro-organisms are also acclimatised, that is to say that they have a physiological state which permits them to develop a fermentative metabolism as soon as they are introduced into musts with high concentrations of alcohol and/or acid, without a large reduction in activity. In other words, the acclimatised and immobilised living micro-organisms of the preparation according to the invention have the ability to start fermentation, directly after a rehydration step, in a must which has an alcoholic degree of between 5 and 15° and a pH of between 2.8 and 4.0.

In addition, the acclimatised and immobilised living micro-organisms of the preparation according to the invention retain these properties for several months, and this has a decisive advantage for their commercial exploitation. For this, it is sufficient to preserve the beads containing the partially dehydrated micro-organisms in packaging sealed from water vapour, preferably kept at a relatively low temperature, conventionally about 4° C. In these conditions, the micro-organisms are preserved in an excellent state for at least six months in storage.

The present invention also relates to a method of obtaining a preparation of micro-organisms, such as described above, comprising the following operations:

a)—acclimatising said micro-organisms to alcohol and/or to acid,

b)—immobilising said micro-organisms in polymer beads, and

c)—partially dehydrating said beads,

which operations are carried out in the chronological order [a) then b) then c)], or indeed [b) then a) then c)].

According to the present invention, the acclimatisation and immobilisation steps may be carried out in any order but they must, of necessity, precede the partial dehydration step.

According to a preferred embodiment of the present invention, the micro-organisms prepared thereby are yeasts, preferably of the Saccharomyces genus, which are immobilised in beads of reticulated polysaccharides, preferably in an alginate gel, in a proportion of between 1 and 50%, preferably between 5 and 20% dry weight, relative to said polymer.

At the start, a culture of micro-organisms is obtained in a conventional culture medium. Then, with these micro-organisms, either the acclimatisation or the immobilisation of the micro-organisms is carried out first, the techniques used for each operation being independent of each other as well as of the order selected for carrying them out.

It is possible, in a variant of the implementation, not to use the micro-organisms obtained by culture immediately, but to preserve them in dried form for the desired period. In this case, care will be taken to rehydrate them according to one of the methods well-known to professionals, before carrying out either the acclimatisation or the immobilisation of the micro-organisms.

The techniques relating to the immobilisation of micro-organisms are known. For example, the methods described in the patents previously cited can be used. In general, a polymer solution containing the micro-organisms to be coated is reticulated, then the matrix obtained is split by mechanical means, most often by crushing, to form polymer beads containing the micro-organisms trapped thereby. Another technique currently used consists of passing the micro-organisms, in suspension in a polymer solution, through a nozzle to form drops, which are put in contact with a gelifying agent in a hydrophobic phase under agitation. According to one particular aspect of the invention, the micro-organisms are immobilised in a polymer matrix capable of forming a gel which is insoluble in wine, such as alginate, according to a known method (such as, for example, that described by DIVIES et al, in Proc. Colloque SFR, Compiègne, 1979).

The techniques relating to acclimatisation are also well-known. In general, dried micro-organisms are incubated in a solution which has an alcohol or acidity degree comparable with that of the medium in which they are required to act. For example, in the case of yeasts intended for reactivating the fermentation of an alcoholic medium, the dry yeasts are incubated for 30 minutes at 37° C. in a wine with a 10° alcohol strength with an admixture of saccharose. Then the yeasts are recovered, for example by centrifugation, and they are placed in suspension in an alginate solution ready for the immobilisation step.

If the yeasts are already in the immobilised form, rehydration is carried out, then incubation as before. The drying step concludes the operation.

The step of partial dehydration may be carried out by one of the following techniques: lyophilisation, drying on a fluidised bed, using a drying cupboard, until an activity of the water Aw of between 0.1 and 0.5, preferably between 0.3 and 0.4, is obtained in said polymer beads. These techniques are selected because they permit the level of humidity reached to be monitored, that is to say, controlled dehydration is achieved. Beads of dry appearance are obtained, but they contain a small proportion of humidity so as to maintain the viability of the micro-organisms.

According to one particular procedure for implementing the invention, the partially dehydrated beads containing the acclimatised micro-organisms are preserved in packaging sealed from water vapour, which is preferably kept at a relatively low temperature, preferably about 4° C. In these conditions, the micro-organisms are preserved in an excellent state for several months in storage.

Thus prepared and possibly stored, the preparations of micro-organisms according to the invention may be used immediately or within a period of several months, directly after simple rehydration and without an acclimatisation step, to reactivate interrupted fermentation processes, in particular to reactivate interrupted alcoholic fermentation processes, such as the fermentation of red or white wines, of hydromel or of any other drink prepared by alcoholic fermentation.

Other characteristics and advantages of the invention will appear in the light of the non-limiting examples hereinafter.

### EXAMPLES

### Example 1

### Preparation of Semi-Humid Alginate Beads Containing Immobilised Saccharomyces cerevisiae Yeasts, Previously Acclimatised to Alcohol

In this Example, yeasts previously acclimatised to alcohol are immobilised in beads prepared from a solution of sodium alginate. The strain of yeast Saccharomyces cerevisiae L43®, marketed by the Lallemand company (France), is used. The acclimatisation step consists of incubating the yeasts for 30 minutes at 370 C at the rate of 100 g dry yeasts for 1 litre liquid, in a wine with a 100 alcohol strength and with an admixture of 100 g saccharose per litre. After incubation, the yeasts are recovered by centrifugation and re-suspended in the 4% sodium alginate solution.

This solution then passes into a system of tubes subjected to vibrations which permit drops to be formed. These drops are then gelified by contact with a 0.2 M solution of calcium chloride. The contact time is 30 minutes.

The beads formed thereby are washed by immersion for 10 minutes in deionised water. The beads are then partially dried on a fluidised bed until a water activity Aw of between 0.3 and 0.4 is obtained. The drying temperature is less than or equal to 40° C. The diameter of the beads obtained is between 2 and 4 mm. After quality controls, the yeasts are packaged and stored at 40 C before use.

### Example 2

### Acclimatisation of Previously Immobilised Yeasts

Yeasts obtained by the rehydration in water of active dry L2226® yeasts, marketed by the Lallemand company (France), are immobilised then acclimatised. Alginate beads are prepared, as in Example 1 above, with non-acclimatised yeasts. The beads obtained are then recovered by sieving and incubated for 30 minutes at 37° C. in a wine with a 10° alcohol strength and an admixture of 100 g saccharose per litre. After sieving and washing by a 9 g/l solution of sodium chloride, the beads are subjected to the drying step in the conditions described in Example 1. After quality controls, the yeasts are packaged and stored at 4° C. before use.

### Example 3

### Stability of the Yeasts in the Course of Storage

In order to estimate the stability of the yeasts, the activity of beads containing the Saccharomyces cerevisiae yeast is measured at time 0 (just after the beads are produced) and after being preserved for 6 months at 4° C. The following monitoring is carried out:

30 grams of beads prepared according to Example 1 above are placed in a 50 g/l glucose solution (volume 100 ml) at 37° C.

The solution is kept at 37° C., and the evolution of the concentration of the sugar is monitored over time.

In the two samples (t=0 and t=6 months), a sugar concentration lower than 2 g/l corresponding to the end of the fermentation is obtained after 2 hours. The end of the fermentation is obtained after the same incubation time. The immobilised yeasts, prepared according to Example 1, are therefore stable during at least 6 months of storage at 4° C.

### Example 4

### Application to White Wine

The white wine used in this Example is a Sauvignon wine which has the following characteristics:

acquired degree of alcohol at the moment of inoculation with the beads: 12.75°

residual sugars: 14.64 g/l

The fermentation is re-started by using L43® yeast, marketed by the Lallemand company (France), immobilised in the form of beads obtained according to Example 1, packaged in units of 5 kg. The dose used is 200 g beads per hi wine. For the purposes of the test, the wine in interrupted fermentation is divided into two demijohns of 25 l (procedures 1 and 2) and two vats of 150 hl (procedures 3 and 4).

These procedures are as follows (the temperature is regulated between 18 and 20° C.):

procedure 1. negative control sample: wine not seeded

procedure 2. positive control sample: wine seeded by L43® yeasts, in the form of dry yeast, dosed at 20 g/hl, rehydrated, non-acclimatised

procedure 3. wine seeded with L43® yeasts, in the form of dry yeast, dosed at 20 g/hl, rehydrated, acclimatised to alcohol in accordance with the procedure called I.T.V., which is known by the person skilled in the art (duration of the rehydration and acclimatisation phases: 7 days)

procedure 4. wine seeded with immobilised L43® yeasts, obtained according to Example 1, prepared in accordance with the following procedure:

Preparation of immobilised L43® yeasts (procedure 4):

a) 30 kg bagged beads are immersed, at a temperature of 37° C., in a vat containing a solution of 60 l water containing 600 g glucose/fructose (50/50) and 480 g NaCl.

b) After 30 minutes, the bags are removed and soaked, in the same conditions, in a new solution identical to the previous solution.

c) Subsequently, the bags are immersed in the vat containing the wine, the alcoholic fermentation of which has to be reactivated.

For each of the 4 procedures, the evolution of the sugar level as a function of time is monitored. The results of this monitoring are shown in the following Table 1:

TABLE 1

DAYS /
RESIDUAL SUGARS (g/l)

| | Procedure 1 | Procedure 2 | Procedure 3 | Procedure 4 |
|---|---|---|---|---|
| 0 | 14.64 | 14.64 | 14.64 | 14.64 |
| 1 | | | | 13.66 |
| 4 | | | | 12.50 |
| 5 | | | | 12.10 |
| 7 | 14.64 | 14.67 | 14.64 | |
| 9 | | | 12.46 | |
| 10 | | | | 8.88 |
| 11 | | | 10.54 | 8.00 |
| 14 | 16.62 | 14.64 | 8.66 | 5.71 |
| 17 | | | | 4.34 |
| 20 | | | 5.55 | 2.48 |
| 22 | 14.65 | 14.60 | 3.07 | |
| 24 | | | | 1.80 |
| 26 | 14.64 | 14.62 | 2.00 | |

It is ascertained that the method according to the present invention (procedure 4) permits fermentation to be completed at the end of a shorter period of time than that observed with the same yeasts acclimatised according to the usual method (procedure 3), all other things being equal. In neither case is an increase in the volatile acidity observed.

Thus, the method of use according to the present invention has two substantial advantages:

it permits 2 days to be saved for the total duration of the fermentation operation,

it takes 1 hour, and no longer 7 days, to prepare and monitor the vat base.

### Example 5

### Application to Red Wine

The red wine used in this Example is a wine of the Merlot vine which has the following characteristics:

acquired degree of alcohol at the moment of inoculation with the beads: 99.9 g ethanol/I

pH: 3.2

residual sugars: 19.29 g/l

The fermentation is re-started by using L2226® yeast, marketed by the Lallemand company (France), immobilised in the form of beads obtained according to Example 2, packaged in units of 5 kg. The dose used is 200 g beads per hi wine. For the purposes of the test, the wine in interrupted fermentation is divided into two demijohns of 25 l (procedures 1 and 2) and two vats of 5 hl (procedures 3 and 4).

These procedures are as follows (the temperature is regulated between 20 and 25° C.):

procedure 1. negative control sample: wine not seeded,

procedure 2. positive control sample: wine seeded by L2226® yeasts, in the form of dry yeast, dosed at 20 g/hl, rehydrated, non-acclimatised,

procedure 3. wine seeded with L2226® yeasts, in the form of dry yeast, dosed at 20 g/hl, rehydrated, acclimatised to alcohol in accordance with the I.T.V. procedure, which is known by the person skilled in the art (duration of the rehydration and acclimatisation phases: 6 days),

procedure 4. wine seeded with immobilised L2226® yeasts, obtained according to Example 2, and prepared in accordance with the procedure described hereinafter:

Preparation of immobilised yeasts (procedure 4):

a) 1 kg bagged beads is immersed, at a temperature of 37° C., in a solution of 2 l water containing 20 g glucose/fructose (50/50) and 16 g NaCl.

b) After 30 minutes, the bags are removed and soaked, in the same conditions, in a new solution identical to the previous solution.

c) Subsequently, the bags are immersed in the vat containing the wine, the alcoholic fermentation of which has to be re-activated.

For each procedure, the evolution of the sugar level as a function of time is monitored. The results of this monitoring are shown in the following Table 2:

TABLE 2

| DAYS / | | | |
|---|---|---|---|
| Procedure 1 | Procedure 2 | Procedure 3 | Procedure 4 |
| residual sugars (g/l)/ | residual sugars (g/l)/ | residual sugars (g/l)/ | residual sugars (g/l)/ |
| ethanol (g/l) | ethanol (g/l) | ethanol (g/l) | ethanol (g/l) |

| | | | | |
|---|---|---|---|---|
| 0 | 19.3/99.9 | 19.3/99.9 | 19.30/99.90 | 19.30/99.90 |
| 2 | Unchanged | Unchanged | Unchanged | 18.87/101.48 |
| 4 | Unchanged | Unchanged | Unchanged | 16.90/101.93 |
| 5 | Unchanged | Unchanged | Unchanged | 14.38/102.36 |
| 6 | Unchanged | Unchanged | Unchanged | 13.73/102.37 |
| 7 | Unchanged | Unchanged | 19.29/100.66 | — |
| 11 | Unchanged | Unchanged | 16.93/101.35 | 4.20/104.86 |
| 12 | Unchanged | Unchanged | 11.63/102.41 | 2.76/105.29 |
| 13 | Unchanged | Unchanged | 7.46/103.53 | 2.21/105.31 |
| 14 | Unchanged | Unchanged | 3.81/104.91 | — |
| 15 | Unchanged | Unchanged | 1.83/105.39 | — |

It is ascertained that the method according to the present invention (procedure 4) permits fermentation to be completed at the end of a shorter period of time than that observed with the same yeasts acclimatised according to the usual method (procedure 3), all other things being equal. In neither case is a rise in the volatile acidity observed.

Thus, the method of application according to the present invention has two substantial advantages:

1. it permits a saving of 2 days for the total duration of the fermentation operation,

2. it takes 1 hour, instead of 6 days, to prepare and monitor the vat base.

### Example 6

### Application to Hydromel

The hydromel used in this Example is derived from a mixture of 300 g honey/1 litre water, the aim being to manufacture sweet hydromel with an alcohol strength of 12.5%. When the fermentation was interrupted, it had the following characteristics:

acquired degree of alcohol at the moment of inoculation with the beads: 10.5% alcohol

total residual sugars: 87.5 g/l

residual sugars to be fermented: 34 g/l

The fermentation was re-started by using EC 1118® yeast, marketed by the Lallemand company (France), immobilised in beads of calcium alginate obtained according to Example 1, packaged in units of 200 g. The dose used is 200 g beads for 100 l hydromel.

For the purposes of the test, the hydromel in interrupted fermentation is divided into two demijohns of 50 l (procedures 1 and 2). These procedures are as follows (the temperature is regulated between 20 and 25° C.):

procedure 1. hydromel seeded with EC1118® yeast, immobilised and prepared in accordance with the procedure described hereinafter:

a) a bag of 200 g of beads is immersed, at a temperature of 37° C., in a solution of 400 ml water containing 4 g glucose/fructose (50/50) and 3.2 g NaCl.

b) then, after 30 minutes, the bag is removed and soaked, in the same conditions, in a new solution identical to the previous solution.

c) subsequently, the bag is immersed in the vat containing the hydromel, the alcoholic fermentation of which has to be reactivated.

procedure 2. hydromel seeded with EC1118® yeast, in the form of dry yeasts, dosed at 20 g/hl, rehydrated, acclimatised to alcohol in accordance with the I.T.V. procedure adapted to hydromel (duration of the rehydration and acclimatisation phases: 4 days).

The evolution of the sugar level as a function of time is monitored for each procedure. The results of this monitoring are shown in the following Table 3:

TABLE 3

DAYS /
RESIDUAL SUGARS (g/l)

| | Procedure 1 | Procedure 2 |
|---|---|---|
| 0 | 87.50 | 87.50 |
| 1 | 86.00 | 85.00 |
| 4 | 75.00 | 72.50 |
| 5 | 71.50 | 68.50 |
| 7 | 66.20 | 64.00 |
| 9 | 58.50 | 59.50 |
| 11 | 50.00 | 54.70 |
| 14 | 36.70 | 46.00 |
| 20 | 36.50 | 36.20 |

It is ascertained that the method according to the present invention (procedure 1) permits fermentation to be completed at the end of a shorter period of time than that observed with the same yeasts acclimatised according to the usual method (procedure 2), all other things being equal.

Thus, the method of use of the present invention has two substantial advantages:

1. it permits a saving of almost 6 days for the total duration of the fermentation operation;

2. it takes 1 hour, instead of 4 days, to prepare and monitor the vat base.

1. Preparation of living micro-organisms, which are acclimatised to alcohol and/or to acidity and immobilised in partially dehydrated polymer beads.

2. Preparation according to claim 1, characterised in that said living micro-organisms, acclimatised and immobilised, have the ability to start fermenting, directly after a rehydration step, in a must which has an alcoholic degree of between 5 and 15° and a pH of between 2.8 and 4.0.

3. Preparation according to claim 2, characterised in that said living micro-organisms, acclimatised and immobilised, have the ability to start fermenting, after a preservation period lasting between 1 day and at least six months, directly after a rehydration step, in a must which has an alcoholic degree of between 5 and 15° and a pH of between 2.8 and 4.0

4. Preparation according to claim 1, characterised in that said micro-organisms are yeasts, preferably of the Saccharomyces genus.

5. Preparation according to claim 1, characterised in that said polymer beads are made up of reticulated polysaccharides, preferably of an alginate gel.

6. Preparation according to claim 1, characterised in that, in said polymer beads, the activity of the water is between 0.1 and 0.5, preferably between 0.3 and 0.4.

7. Preparation according to claim 1, characterised in that the size of said beads is between 0.1 and 5 mm, preferably between 1 and 3 mm.

8. Preparation according to claim 1, characterised in that said beads contain said micro-organisms in a proportion of between 1 and 50%, preferably between 5 and 20% dry weight, relative to said polymer.

9. Method of obtaining a preparation of micro-organisms according to claim 1, comprising the following operations:
a)—acclimatising said micro-organisms to alcohol and/or to acid,
b)—immobilising said micro-organisms in polymer beads, and
c)—partially dehydrating said beads,
which operations are carried out in the chronological order [a) then b) then c)], or indeed [b) then a) then c)].

10. Method according to claim 9, in which said micro-organisms are yeasts, preferably of the Saccharomyces genus.

11. Method according to claim 9, in which said polymer beads are made up of reticulated polysaccharides, preferably of an alginate gel.

12. Method according to claim 9, in which said micro-organisms, immobilised in said polymer beads, are in a proportion of between 1 and 50%, preferably between 5 and 20% dry weight, relative to said polymer.

13. Method according to claim 9, in which said partial dehydration is effected by one of the following techniques: lyophilisation, drying on a fluidised bed, using a drying cupboard, until an activity of the water of between 0.1 and 0.5, preferably between 0.3 and 0.4, is obtained in said polymer beads.

14. Preparation of micro-organisms capable of being obtained by a method according to one of claims 9 to 13.

15. Use of a preparation of micro-organisms according to one of claims 1 to 8 for the re-starting of interrupted fermentation processes.

16. Use of a preparation of micro-organisms according to one of claims 1 to 8 for the re-starting of an interrupted alcoholic fermentation.
